(19) United States
(12) Patent Application Publication
(10) Pub. No.: US 2026/0157349 A1
(43) Pub. Date: Jun. 11, 2026

(54) IDENTIFYING SEPARATION-RELATED PROBLEMS IN PETS

(71) Applicant: Société des Produits Nestlé S.A., Vevey (CH)

(72) Inventors: Lorna Winter, London (GB); Daniel Mills, Lincolnshire (GB); Anna Zamansky, Haifa (IL); Luciana Santos de Assis, Lincolnshire (GB)

(73) Assignee: Société des Produits Nestlé S.A., Vevey (CH)

(21) Appl. No.: 19/179,886

(22) Filed: Apr. 15, 2025

Related U.S. Application Data
(60) Provisional application No. 63/648,823, filed on May 17, 2024.

(51) Int. Cl. A01K 29/00; G06N 20/00

(52) U.S. Cl. CPC A01K 29/007; G06N 20/00

(57) ABSTRACT

The present disclosure relates to systems, methods, and program applications for identifying separation-related problems in a pet. The methods, for example, can include identifying the presence or absence of multiple behavioral signs exhibited by a pet where each of the multiple behavioral signs are given a sign score based on binary annotations representing either the presence or the absence of each of the behavioral signs, and grouping subsets of the multiple behavioral signs into one of multiple principal component behavioral groupings using the binary annotations to generate principal component scores for each of the multiple principal component behavioral groupings. Methods can also include using one or more machine-learning algorithms under the control of at least one processor for accessing and correlating the principal component scores for each of the multiple principal component behavioral groupings with a population cluster associated with a type of separation-related problem.

### CROSS REFERENCE TO RELATED APPLICATION

This application claims priority to U.S. Provisional Application Ser. No. 63/648,823 filed May 17, 2024 the disclosure of which is incorporated in its entirety herein by this reference.

### BACKGROUND

The estimated percentage of dogs globally with separation-related problems (SRP), such as separation anxiety, may be at least about 20% and in some analyses, may be up to about 55%. Separation anxiety is one such issue from which many dogs (and other domesticated pets) suffer. SRPs can be manifest in pets based on certain behaviors, which may be related to frustration and/or boredom, to name a few. A combination of understanding pet behaviors and knowledge about treatment related to SRPs can lead to a healthier and happier pet. Thus, it would be beneficial to provide pet owners or caregivers tools or workflows for identifying and treating pets suffering from one or more SRP, such as separation anxiety.

### BRIEF DESCRIPTION OF THE DRAWINGS

FIG. 1 schematically illustrates example systems of identifying separation-related problems in pets in accordance with the present disclosure;

FIG. 2 is a flow diagram illustrating an example statistical system and method for identifying separation-related problems in pets that includes the use of statistical analysis used for generating a machine-learning workflow that is usable by an end user, e.g., a pet caregiver, in accordance with the present disclosure; and

FIG. 3 is a flow diagram illustrating an example method of generating a machine-learning workflow usable for identifying separation-related problems in a pet in accordance with the present disclosure.

### DETAILED DESCRIPTION

The present disclosure relates to pet health, and more particularly to systems, methods, and computer program products (machine-readable storage medium) that can be used for identifying separation-related problems in pets. Notably, the terms “separation-related problem,” “SRP,” “separation-related disorders,” “separation anxiety,” etc., often refer to the same condition(s) to which a pet may be suffering. These terms may be used interchangeably herein.

In accordance with examples of the present disclosure, a method of identifying separation-related problems in a pet can include identifying the presence or absence of multiple behavioral signs exhibited over a population of pets, wherein each of the multiple behavioral signs can be given a sign score based on binary annotations representing either the presence or the absence of each the behavioral sign. The method can also include grouping subsets of the multiple behavioral signs into one of multiple principal component behavioral groupings using the binary annotations to generate principal component scores for each of the multiple principal component behavioral groupings for at least a plurality of pets across the population of pets, and can further include correlating the principal component scores for each of the multiple principal component behavioral groupings across the plurality of pets. Each pet of the plurality of pets can be assigned to a population cluster associated with a type of separation-related problem for the plurality of pets. The population cluster for each of the plurality of pets can have a better fit for the separation-related problem than other population clusters associated with other types of separation-related problems. The method can also include training an artificial intelligence with one or more machine-learning algorithms under the control of at least one processor using a training dataset loaded on a memory device. The training dataset can be based on the principal component scores related to the plurality of pets as correlated with the population clusters.

In another example, a machine-learning system for identifying separation-related problems in a pet can include at least one processor and at least one memory device including a data store to receive behavioral information related to an individual pet which is entered into a client device by a pet caregiver. The data store can also include instructions that, when executed, cause the system to correlate the behavioral information with a plurality of binary sign scores related to the presence or absence of multiple behavioral signs related to the individual pet, and group subsets of the multiple behavioral signs into one of multiple principal component behavioral groupings to generate principal component scores for each of the multiple principal component behavioral groupings. The instructions can also, when executed, cause the system to correlate the multiple principal component scores for each of the multiple principal component behavioral groupings to assign the individual pet to a population cluster associated with a type of separation-related problem. The population cluster can be selected by having a better fit for the separation-related problem than other population clusters associated with other types of separation-related problems.

In another example, a non-transitory machine-readable storage medium can have instructions embodied thereon such that when the instructions are executed by one or more processors, the one or more processors correlate the behavioral information with a plurality of binary sign scores related to the presence or absence of multiple behavioral signs related to the individual pet, and group subsets of the multiple behavioral signs into one of multiple principal component behavioral groupings to generate principal component scores for each of the multiple principal component behavioral groupings. The one or more processors can also correlate the multiple principal component scores for each of the multiple principal component behavioral groupings to assign the individual pet to a population cluster associated with a type of separation-related problem. The population cluster can have a better fit for the separation-related problem compared to other population clusters associated with other types of separation-related problems. For example, the population cluster can be correlated higher to a specific separation-related problem than other types of separation-related problems. In some examples, the one or more processors can also report the population cluster, the separation-related problem, or both to the pet caregiver at the client device.

In these examples related to the methods, systems, and machine-readable storage media, a variety of details can be considered. For example, a portion or all of the binary annotations for at least some of the behavioral signs can be scored 0 for the absence of the behavioral signs and 1 for the presence of the behavioral signs. In some examples, a portion of the binary annotations for at least one of the behavioral signs can be scored −1 for the presence of a positive behavioral sign and 1 the presence of a negative behavioral sign. There may be any number of behavioral signs used, but in some examples, there may be at least 50 behavioral signs that are considered for the grouping of subsets of the multiple behavioral signs into one of the multiple principal component behavioral groupings.

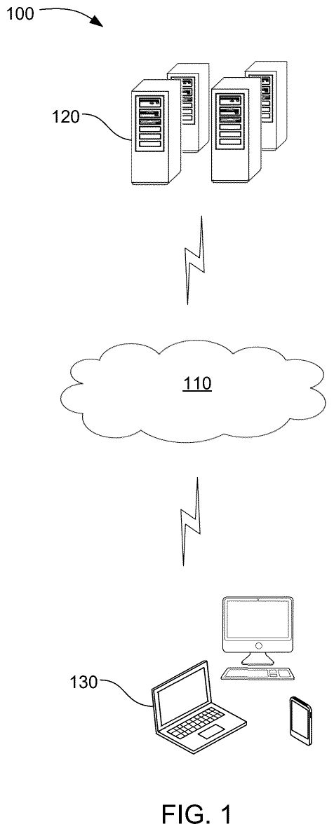

Turning now to the drawings, FIG. 1 schematically illustrates system 100 for identifying separation-related problems in pets. The system can include a client device(s) 130 and analysis server(s) 120 in communication via a network 110. In this example, identifying the presence or absence of multiple behavioral signs may be carried out by a pet caregiver answering inquiries about the pet using a client device(s) connected to the analysis server(s) which includes the capability for machine-learning.

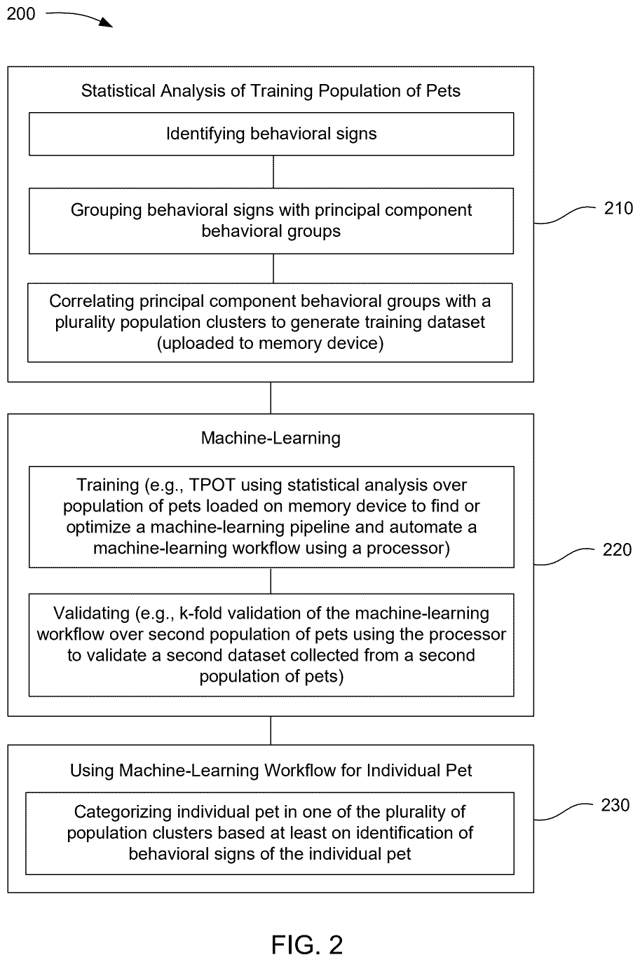

The methods described herein (and systems utilizing these methods) can be implemented as illustrated in some examples based on the flow diagram shown at 200 in FIG. 2. This example method can include carrying out a statistical analysis 310 of a training population of pets, e.g., dogs, by identifying behavioral signs within the population of pets, grouping behavioral signs with principal component behavioral groups, and correlating the principal component behavioral groups with a plurality of population clusters to generate a training dataset. The training dataset can be loaded on a memory device and using a processor, can undergo training, e.g., TPOT, and validation, e.g., k-fold validation, using machine-learning 320 to generate a machine-learning workflow that generates accurate categorization in population clusters using information from the statistical analysis. Once trained and validated, in some examples, the artificial intelligence including the machine-learning workflow can be used to categorize an individual pet in one of the plurality of population clusters based at least on identification of behavioral signs of the individual pet. Notably, the machine-learning is described in this instance as being used for training and validation of a population (or cohort) of dogs for purposes of training and a second population (or cohort) of dogs for validation. However, once trained and validated, the machine-learning can likewise be trained to utilize the information collected on a client device (entered by pet caregiver, such as from a questionnaire to determine behavioral signs), and the memory device and processor across a network (see FIG. 1) can automatically group the behavioral signs into subsets correlated with the appropriate principal component behavioral groupings and behavioral cluster, which can be reported to the pet caregiver over the network to the client device.

Though any pet can benefit from this machine-learning technology, in some examples, the pet can be a dog. In these examples, the principal component behavioral groupings can include one or more, two or more, three or more or all of the groupings selected from exit frustration (Ef), redirected frustration (Rf), social panic (Sp), elimination (E), reactive communication (Rc), immediate frustration (If), or noise sensitivity (Ns). In some examples, the population clusters can each be associated with a unique separation-related problem. For example, with dogs, the population clusters can include one or more (or all four) population clusters selected from exit frustration, redirected reactive, reactive inhibited, or boredom. In still further detail, correlating the principal component scores with the population cluster can include exclusion of at least one behavioral cluster as a possibility, assigning a closest source feature represented by the principal component scores of the multiple principal component behavioral groupings using metric learning, or both. Training and/or validating the one or more machine-learning algorithms can include or be based on a linearly fitted model utilizing a stochastic gradient decent algorithm, the behavioral signs collected from a first plurality of pets of a training cohort and the behavioral signs collected from a second plurality of pets of a validation cohort, the use of tree-based pipeline optimization and k-fold cross-validation, or a combination thereof.

It is noted that when discussing examples related to the methods of identifying separation-related problems in a pet, the machine-learning systems for identifying separation-related problems in a pet, and the non-transitory machine-readable storage media described herein, such discussions can be considered applicable to other related examples that are not specifically explicitly discussed in the context of that example. For example, when discussing “behavioral signs” in the context of the methods, such disclosure can also be related to systems and/or machine-readable storage media, and vice versa. Furthermore, terms used herein will have their ordinary meaning in the relevant technical field unless specified otherwise. In some instances, there are terms defined more specifically throughout the specification, with a few more general terms included at the end of the specification. These more specifically defined terms have the meaning as described herein.

Separation-related problem (SRP) datasets can be generated based on any number of samples of multiple pets of a specific animal type, e.g., several hundred dogs, where behavioral signs are identified and then categorized into principal component behavioral groupings, followed by the grouping of pets within one of a plurality of population clusters. For example, in the case of dogs, specific behavioral signs can be used to identify principal component behavioral groupings. In this instance, seven (7) principal component behavioral groupings (each including multiple behavioral signs within one or more principal component behavioral groupings) may be identified. As an example, principal component behavioral groupings for dogs can include exit frustration (Ef), redirected frustration (Rf), social panic (Sp), elimination (E), reactive communication (Rc), immediate frustration (If), and noise sensitivity (Ns). Thus, the principal component behavioral groupings each include a collection of behavioral signs that are often related from a behavioral perspective.

These seven principal component behavioral groupings are identified in Table 1 below, along with multiple behavioral signs correlating therewith. Notably, other principal component behavioral groupings and/or correlated behavioral signs may be identified for dogs or for other pets without departing from the teachings of the present disclosure. This example is merely used to illustrate the technology described herein.

TABLE 1
Principal Component Behavioral Groupings

| Principal | | |
|---|---|---|
| Component | | |
| Behavioral | Behavioral | |
| Groupings | Sign # | Behavioral Signs |
| Exit | 1 | Destruction in your absence of big objects (furniture, |
| Frustration | | windows, doors, door frames, other exit points from |
| (Ef) | | house) |
| | 2 | Destruction in your absence of doors/around doors |
| | 3 | Destruction in your absence of the main exit door of the room |
| | | where the dog was left |
| | 4 | Destruction in your absence of the main exit door, where the |
| | | dog was left, when it was closed |
| | 5 | Destruction in your absence of door frame next to where the |
| | | door opens |
| | 6 | Destruction in your absence of door itself next to where it |
| | | opens |
| | 7 | Destruction in your absence on or around door handle |
| | 8 | Destruction in your absence of floor nearby the place where |
| | | the door opens |
| | 9 | Destruction in your absence of house structure (holes in wall, |
| | | torn up linoleum) |
| | 10 | In your absence, your dog destroys using his/her claws |
| Redirected | 11 | Destructiveness in your absence |
| Frustration | 12 | Destruction in your absence of medium-sized items (e.g. |
| (Rf) | | pillows) |
| | 13 | Destruction in your absence of clothing |
| | 14 | In your absence, your dog destroys using his/her mouth |
| | 15 | Takes objects and destroys them when confined in a room |
| | | without access to you |
| | 16 | Takes objects and destroys them while left home alone |
| | | without human company |
| Social | 17 | Vocalizes without human company |
| Panic | 18 | Vocalizes frequently when left home alone for at least 1 h |
| (Sp) | | (most times or always, i.e. 66 to 100% of time) |
| | 19 | Vocalizes frequently when separated from you within the |
| | | home for at least 1 h - most times or always (i.e. 66 to |
| | | 100% of time) |
| | 20 | Whines frequently without human company - always (i.e. |
| | | 100% of the time) |
| | 21 | Barks frequently without human company - always (i.e. |
| | | 100% of the time) |
| | 22 | Whines when getting ready to leave the house for any daily |
| | | activity |
| | 23 | Paces when getting ready to leave the house for any daily |
| | | activity |
| | 24 | Whines when getting ready to leave the house at an unusual |
| | | time |
| | 25 | Paces when getting ready to leave the house at an unusual |
| | | time |
| | 26 | Vocalizes during short separation period from you at home |
| | | when nobody else is there |
| | 27 | Looks anxious during short separation period from you at |
| | | home when nobody else is there |
| | 28 | Vocalizes after you have stepped outside the house |
| | 29 | Bites and/or claws the door/window/crate after you have |
| | | stepped outside the house |
| | 30 | Becomes distressed frequently in anticipation of you trying to |
| | | withdraw from his/her presence - often or always (i.e. 66 |
| | | to 100% of the time) |
| | 31 | Becomes restless, agitated or pacing frequently when |
| | | confined or left home alone - often or always (i.e. 66 to |
| | | 100% of the time) |
| Elimination | 32 | House soiling (urine) in your absence |
| (E) | 33 | House soiling (feces) in your absence |
| | 34 | Urinates in inappropriate places in the home when alone or |
| | | shut in somewhere |
| | 35 | Urinates in inappropriate places even when left alone or shut |
| | | in somewhere |
| | 36 | Urinates when alone that started only after 6 months of age |
| | 37 | Defecates in inappropriate places in the home when alone or |
| | | shut in somewhere |
| | 38 | Defecates in inappropriate places even when alone or |
| | | confined |
| | 39 | Defecates when alone that started only after 6 months of age |
| | 40 | House soiling frequently when left home alone for at least |
| | | 1 h - most times or always (i.e. 66 to 100% of time) |
| | 41 | House soiling frequently when alone in the house that |
| | | happened only after 6 months of age - almost every day or |
| | | at least once/day |
| Reactive | 42 | Barks and/or whines when somebody comes to the door |
| Communication | 43 | Wags tail when somebody comes to the door |
| (Rc) | 44 | Barks when doorbell rings or someone knocks on the door |
| | 45 | Wags tail when he/she is in the car and an unfamiliar |
| | | person/dog comes near |
| | 46 | Barks and/or whines when he/she is in the car and an |
| | | unfamiliar person/dog comes near |
| | 47 | Barks and/or whines when he/she sees an unfamiliar |
| | | person/dog come near the home but can't reach them due |
| | | to some barrier (e.g. gate, lead) |
| Immediate | 48 | Growls when he/she sees (from the window) you chatting to |
| Frustration | | your neighbor on your driveway for a few minutes after |
| (If) | | arriving home |
| | 49 | Bites or tries to bite you when you try to put him/her on the |
| | | lead earlier than normal after a run in the park or when |
| | | he/she gets a significantly shorter walk than usual |
| | 50 | Bites or tries to bite you when he/she is not allowed to play |
| | | free in the park or do some other usual activity |
| Noise | 51 | Panics and starts to destroy things when hears a sudden loud |
| Sensitivity | | noise (e.g. car backfire, objects falling) |
| (Ns) | 52 | Panics and starts to destroy things when hears fireworks |
| | 53 | Panics and starts to destroy things when hears screeches/ |
| | | whistles |
| | 54 | Panics and starts to destroy things when hears thunderstorms |

In accordance with Table 1, behavioral signs of individual pets of a population can be noted and used to determine their principal component behavioral grouping(s) based on a structure of variation in behavioral presentation. For example, a hierarchical agglomerative cluster analysis can then be used to determine sub-populations of dogs (or other pets) from the full population used to generate data. Essentially, each dog can be given a sign score from a binary system, e.g., ±1, 0/1, etc., for each of the behavioral signs which is then divided by a total maxim. That value can then be divided by the maximum total of possible behavioral signs for each of the principal component behavioral groupings. This can result in calculating standardized principal component scores (one for each principal component category—seven in this instance) for each dog, These values can then be used to calculate a value suitable to categorize each dog into one of four (4) population clusters for hierarchical agglomerative cluster analysis.

As a note, reactive communication (Rc) in this example can be scored differently than the other six (6) principal component behavioral groupings due to the way this principal component behavioral grouping operates. For each of the following six behavioral groupings, namely exit frustration (Ef), redirected frustration (Rf), social panic (Sp), elimination (E), immediate frustration (If), and noise sensitivity (Ns), the principal component scores are generated by totaling the values of the behavioral signs from each principal component behavioral grouping. Regarding reactive communication (Rc), when two of the behavioral signs relating to tail wagging are present, those behavioral signs are given a negative score, e.g., minus one (−1 or −Rc) for “wags tail when somebody comes to the door” and/or minus one (−1 or −Rc) for “wags tail when he/she is in the car and an unfamiliar person/dog comes near.” The other four (4) behaviors relate to barking, are each given a positive score when present. Notably, the negative score for tail wagging reduces the score of reactive communication. As an example, a total score ranging from −2 to +4 may be obtained for the reactive communication principal component behavioral grouping, e.g. if the dog shows “wagging tail when a person is at the door” and barks when “the doorbell rings” and “inside a car when an unfamiliar person/dog approaches” the total would be: −1+1+1=+1. As another example, if a dog were to exhibit tail wagging in both situations, e.g., “when a person is at the door” and “when someone approaches the car,” but shows none of the other Rc behavioral signs, the score would be: −1+(−1)=−2, making this a less likely principal component behavioral grouping appropriate for that particular pet.

Based on the principal component behavioral grouping scoring, these behavioral groupings can then be combined as described above to determine what population cluster(s) would be most appropriate for that specific pet (based on machine-learning training data previously conducted over a large population of similar pets, e.g., dogs, as described in greater detail herein. In accordance with examples herein, four (4) different population clusters representing different types of separation-related problems (SRPs) can be used. It is noted that four are used in the present disclosure by example only for use with dogs, but a greater number or fewer number of population clusters may be used or identified for use in accordance with the present disclosure. Furthermore, there may be different behavioral signs and/or principal component behavioral groupings for other types of pets, such as cats for example.

As illustrated in Table 2 by example, four (4) behavioral clusters are provided for dog populations, which have been found to provide adequate behavioral differences to establish different distinct separation-related problem sub-populations in dogs. These population clusters are based on patterns of behavioral signs and principal component behavioral groupings. For example, each of these four population clusters for dogs can represent a different set of problematic behaviors exhibited by dogs when they are left alone. In accordance with this, the four population clusters are shown and are labeled as Cluster A, Cluster B, Cluster C, and Cluster D below.

TABLE 2
Population Clusters of Separation-related Problems

| Dog Classification | Behavior-related SRP |
|---|---|
| Cluster A | Exit Frustration |
| Cluster B | Redirected Reactive |
| Cluster C | Reactive Inhibited |
| Cluster D | Boredom |

In more detail regarding the four (4) population clusters described in Table 2, Cluster A represents SRP behavior categorized as exit frustration. This population is characterized by its dog members showing behavioral signs of exit frustration at a relatively high level (on average a little more than 7 of the 10 behavioral signs included in this principal component behavioral grouping), social panic and redirected frustration (at relatively high levels—on average about 9/15 and about 4/6 behavioral signs, respectively), but no behavioral signs of immediate frustration towards the owner. Without being bound by any particular theory, one explanation for the principal component behavioral grouping profile of this dog population cluster is that these dogs find being separated aversive (social panic behavioral signs) and try to go after the owner (exit frustration). However, because they are unable to do so due to barriers within the home, they struggle to find an alternative way of coping (redirected frustration). They do not appear hostile to their owners (immediate frustration).

Cluster B represents SRP behavior categorized as redirected reactive. This population is characterized by its dog members showing redirected frustration and reactive communication at relatively high levels (on average, about 4-5/6 behavioral signs and about +3 from the range −2 to +4 of behavioral signs, respectively), nearly all showing behavioral signs of social panic (on average, about 6/15 behavioral signs), with about three quarters showing behavioral signs of exit frustration, but at a low level (on average just about one sign for those that show any of the behavioral signs in this principal component behavioral grouping). Although immediate frustration towards the owner is a rare phenomenon in SRP, this is one of the dog population clusters where this behavior is featured. Without being bound by any particular theory, one explanation for the principal component behavioral grouping profile of this dog population cluster is that these dogs are agonistically reactive to external events (reactive communication), and often try to get at these stimuli as an immediate response (exit frustration) but because they are unable to do so due to the barriers within the home, they remain highly aroused and struggle to find an alternative way of coping (redirected frustration). Accordingly, these dogs find being separated from the owner aversive (social panic behavioral signs), with the rare instances of aggression towards the owner (immediate frustration) being indicative of a more general agonistic reactivity.

Cluster C represents SRP behavior categorized as reactive inhibited. This population is characterized by its dog members showing behavioral signs of reactive communication at relatively high levels (about +3 from the range −2 to +4 of behavioral signs) and nearly all showing behavioral signs of social panic (at relatively moderate levels −5-6/15 behavioral signs on average). Behavioral signs of exit frustration are rare (<5% of dogs) and redirected frustration uncommon (only about a fifth of cases) and at a low level when it does occur. Although immediate frustration towards the owner is a rare phenomenon in SRP, like Cluster B, this is the other group which tends to exhibit this behavior. Without being bound by any particular theory, one explanation for the principal component behavioral grouping profile of this dog population cluster is that these dogs are reactive to external events (reactive communication), but unlike cluster B, dogs do not typically try to get at these stimuli (exit frustration). This might be, for example, because they are more generally anxious and avoidant than dogs in Cluster B. This would be consistent with the absence of the owner being associated with a loss of social support (social panic) but the dogs perhaps find some safety in the home, reducing their arousal (redirected frustration). The rare instances of aggression towards the owner (immediate frustration) might be indicative of a more extreme nervousness.

Cluster D represents SRP behavior categorized as boredom. This population is characterized by a lack of consistency in behavioral signs across all dog members, although none show behavioral signs of immediate frustration towards the owner; exit frustration is also relatively rare (24% of dogs). Social panic is the most frequently shown group of behavioral signs (78.8%) although the number of behavioral signs is significantly less than the other clusters, with redirected frustration and reactive communication shown by a majority of subjects, albeit the latter with a relatively low number of behavioral signs. Without being bound by any particular theory, one explanation for the principal component behavioral grouping profile of this dog population cluster is that these dogs have learned that being alone is aversive (social panic) due to a lack of stimulation. This leads them to become more reactive to external events as time progresses, which may ultimately relate in redirected frustration, as they cannot escape.

Table 3 below provides an example of how an individual dog may be scored with respect to the principal component behavioral groups and associated behavioral signs. This particular table provides information primarily about when a behavioral cluster can be ruled out as the appropriate cluster, though there are two situations shown where Cluster A is supported as a possibility.

TABLE 3
Likelihood of Population Cluster Based on
Principal Component Behavioral Grouping

| | Total Number | | | |
|---|---|---|---|---|
| Behavioral | of Behavioral | Extremely | | Supportive |
| Grouping | Signs | Unlikely | Unlikely | Of |
| Exit | 0 | A | — | — |
| Frustration | ≤2 | — | A | — |
| (Ef) | ≥5 | — | — | A |
| Redirected | 0 | A, B | — | — |
| Frustration | ≤2 | — | A, B | — |
| (Rf) | ≥3 | — | C | — |
| | 6 | C | D | — |
| Social Panic | 0 | A | — | — |
| (Sp) | ≤4 | — | A | — |
| | ≥11 | — | D | A |
| Elimination | — | — | — | — |
| (El) | | | | |
| Reactive | −2 | B, C | — | — |
| Communication | ≤0 | — | B, C | — |
| (Rc) | ≥2 | — | D | — |
| Immediate | — | — | — | — |
| Frustration | | | | |
| (If) | | | | |
| Noise | ≥2 | — | A, C, D | — |
| Sensitivity | | | | |
| (Ns) | | | | |

In further detail regarding the examples shown in Table 3 above, starting with exit frustration (Ef), if the dog's score is 0, it is extremely unlikely to belong to Cluster A. This means when evaluating other principal component behavioral groupings, evidence for Clusters B, C, and D can be considered as more likely. This process can be carried out for each of the seven (7) principal component behavioral groupings to determine if there is a reasonably clear population cluster to which the pet, e.g., dog, may be appropriate, or in many instances, may be extremely unlikely or unlikely for assignment to a behavioral cluster.

In further detail, if there is low confidence as to what population cluster to assign a dog, a second stage evaluation may be carried out. The second stage evaluation may include bringing forward the information from the initial population cluster data collected (shown by example in Table 3). For example, a specific dog may not clearly fit within one or two of the four population clusters, leaving one or two clusters, respectively, as possibilities for assignment. The second stage evaluation may involve querying the pet caregiver, e.g., dog owner, veterinarian, kennel operator, etc., relating to the likelihood of some of the behavioral signs occurring within a particular population cluster (see Table 2). The second stage evaluation may involve completing the full evaluation, or in some instances, the proper cluster may be ascertainable earlier in the second stage evaluation process.

Regarding individual pets returning results indicating it is extremely unlikely or unlikely to fall within a specific behavioral cluster, again as shown by example in Table 3, typically, a professional judgement is made based on the closest match. However, in accordance with the present disclosure, machine-learning algorithms can be used as a substitute for this professional judgment in some instances. As an illustration of appropriately identifying separation-related problems, focus on some evidence can allow for the exclusion of certain behavioral clusters as possibilities. For example, there are certain behavioral clusters in which certain principal component behavioral groupings typically occur.

Table 4 below provides an example related to the behavioral sign and/or principal component behavioral grouping scores that may be typical for the dogs assigned to each of the four population clusters, e.g., Cluster A, Cluster B, Cluster C, or Cluster D. In this example, Table 4 illustrates at least one sign of a given principal component behavioral grouping as well as a typical number of total behavioral signs from each grouping. This approach is appropriate for six of the seven principal component behavioral groupings, but as previously mentioned, reactive communication (Rc) is scored differently due to the two possible negative values that may be provided for tail wagging that partially negates positive scores that occur for various types of barking activity. More specifically, because reactive communication (Rc) has two behavioral signs that can have a negative value, a range of −2 to +4 for each dog is a possibility. In this instance, if a dog showed a total principal component score of “0” for reactive communication, this could be because the dog indicated no behavioral signs within this principle component behavioral grouping, or because it had the same number of negative and positive behavioral sign scores, e.g. one negative and one positive or two negatives and two positives.

In Table 4, ranges are provided for the total number of behavioral signs that may be typical for dogs falling within a given principal component behavioral grouping. It is noted that these ranges are provided by example only, but still represent a reasonable range of behavioral sign scores for each grouping.

TABLE 4
Behavioral Sign Scores for Principal Component Behavioral
Groupings to Determine Appropriate Behavioral Cluster

| Principal Component | Clus- | Clus- | Clus- | Clus- |
|---|---|---|---|---|
| Behavioral Grouping | ter A | ter B | ter C | ter D |
| Exit Frustration (Ef) | 5-9 | 1-3 | 1-2 | 1-2 |
| Redirected Frustration (Rf) | 3-6 | 3-6 | 1-2 | 1-5 |
| Social Panic (Sp) | 5-13 | 3-10 | 3-11 | 3-9 |
| Elimination (E) | 2-6 | 2-6 | 2-6 | 3-7 |
| Reactive Communication (Rc) | −1-3 | 1-3 | 1-3 | −1-0 |
| Immediate Frustration (If) | 0 | 3 | 2 | 0 |
| Noise Sensitivity (Ns) | 1 | 1-3 | 1 | 1 |

With additional detail regarding Table 4, a typical principal component behavioral score that would be indicative of assigning one of the four behavioral clusters would usually fall within the ranges provided. Scores outside of these ranges could indicate that the cluster being considered is not applicable with respect to that particular principal component behavioral grouping. However, as there are seven (7) principal component behavioral groupings, the preponderance of the evidence may still lead to categorizing a particular pet, e.g., dog, within that particular behavioral cluster, even if the score given for one or two of the behavioral groupings are outliers. Note that the ranges shown in Table 4 represent typical ranges that occur in dogs experiencing separation-related problems. Some specific dogs may fall outside of one or a few of these ranges and yet, the closest identifiable cluster for that particular dog may still be assigned, provided the cluster selected for the dog provides a better fit than the remaining three clusters in this example. When determining the best cluster choice or fit for a dog that does not neatly fall into one of these clusters, clinical expertise can be implemented by a qualified animal behaviorist. The data generated as a result of utilizing clinical expertise can likewise be entered into the system for additional machine-learning going forward.

Table 5 illustrates some reference percentage ranges related to the percentage of dogs within a given cluster that can be categorized within a principal component behavioral grouping. These percentage ranges are based on data collected, but could be narrower or wider than those presented below as additional data is gathered and additional machine-learning occurs through use of the systems and methods described herein. In this example as shown in Table 5, each principal component behavioral grouping indicates the percentage of dogs that exhibited at least one behavioral sign (as outlined in Table 1 above), with the exception of reactive communication, which includes both positive and negative scores as previously described.

TABLE 5
Example Behavioral Sign Percentages Across Population of Dogs

| | % Range Showing One (1) or | | | |
|---|---|---|---|---|
| | more Behavioral Signs | | | |
| Principal Component | Clus- | Clus- | Clus- | Clus- |
| Behavioral Grouping | ter A | ter B | ter C | ter D |
| Exit Frustration (Ef) | 90-100 | 65-80 | 1-10 | 15-30 |
| Redirected Frustration (Rf) | 90-100 | 90-100 | 15-30 | 50-70 |
| Social Panic (Sp) | 90-100 | 90-100 | 85-95 | 70-85 |
| Elimination (E) | 40-60 | 30-50 | 35-55 | 25-40 |
| Reactive Communication (Rc) | 90-100 | 90-100 | 90-100 | 60-80 |
| Immediate Frustration (If) | 0 | >0-1 | >0-1 | 0 |
| Noise Sensitivity (Ns) | 1-4 | 3-6 | 0.5-2 | 1-4 |

Notably, the number of dogs used to generate the data in Table 5 was from a pool of 762 dogs. However, to the extent that there are any outlier samples, such as it may relate to a few atypical dogs exhibiting certain problematic behaviors or otherwise not fitting within one of the categories, those dogs may be excluded for use in the training model for purposes of training the artificial intelligence. In other words, in some instances, it may not be particularly helpful in training the artificial intelligence if there are a minority of samples (or dogs) that skew the data in a way that generates false or shifted clustering parameters that would not benefit the majority of typical dogs. In this particular example, a total of 589 dogs were found to provide the most useful pool of samples for training the artificial intelligence. That is about 77% of the total pool of dogs that could be used initially for training the model. However, once the model is established using the pool of samples representing a majority of the dogs, the outlier samples (or dogs) can then be categorized using the clusters established using the training data. With that said, even if not all of the outlier dogs can be categorized neatly into one of the clusters, still a portion of the outlier dogs will be able to be properly categorized after training the artificial intelligence. It is estimated that at least about 90% of the general population of dogs would benefit from utilizing this system for understanding separation-related problems from which a dog may be suffering.

In further detail regarding Tables 3-5, notably there are high percentages of commonality in some of the behavioral clusters. For example, the dogs of Cluster A tend to always (or almost always) show behavioral signs of exit frustration (Ef), redirected frustration (Rf), and social Panic (Sp). If a specific dog were to show no behavioral signs in any of these principal component behavioral groupings, Cluster A can be excluded as a possible grouping. Likewise, dogs without any behavioral signs of redirected frustration (Rf) and/or reactive communication (Rc) can typically be excluded from being in Cluster B, while dogs without any behavioral signs of reactive communication (Rc) can be excluded from Cluster C. Given the percentages shown for social panic (Sp), it would also be extremely unlikely that a dog with no behavioral signs within that category would be properly categorized in Cluster B.

In further detail, the use of behavioral signs within a principal component behavioral grouping to exclude a dog from a certain cluster where the behavioral sign is rare would more likely be useful only when the figure is at 0%, for example. An illustration of this may be found in the immediate frustration (If) principal component behavioral grouping, where it may be tempting to exclude this behavioral group from Cluster A and Cluster D. In this instance, because this is typically such a rare behavioral grouping (in the current samples), caution should be used if this is the only evidence available regarding behavioral cluster grouping. Similarly, caution can be prudent regarding attributing too much weight to noise sensitivity. In some instances, cleaning of the data prior to running it through the algorithm may be useful. For example, some samples (or pets) can be removed from the dataset by an experienced animal behavioralist when it is clear that the behavior(s) of the pet represent an outlier which would skew the data in a way that would reduce the predictive characteristics of the algorithm(s).

Even though using exclusion or using caution for rare behaviors may not be conclusive, it often assists in determining an appropriate behavioral cluster for a dog by process of elimination and/or weighting rare behaviors appropriately against other available evidence. In carrying out this type of analysis, which can be trained in the context of machine-learning, focus can be place on the behavioral sign scores for each principal component behavioral grouping. As one example, consideration to principal component behavioral groupings should be used only when there is at least one behavioral sign associated with that behavioral grouping. In additional detail, a query may be asked as to whether the score for a specific principal component behavioral grouping is within the typical range of behavioral sign scores, and if it is not, a second query asked may relate to how far from the typical range of scores is the value outside of the typical range. For example, if a dog has a score for exit frustration (Ef) of 9, it is unlikely to belong to Cluster B, Cluster C, or Cluster D, making it most probably a member of Cluster A. On the other hand, if a dog scores 2 for exit frustration (Ef), it may be unlikely that dog belongs in behavioral Cluster A.

In further detail, when the data has been gathered, the entire profile of the principal component behavioral grouping scores can be compared across the various clusters, namely Clusters A-D in this example. The recorded sign scores for each of the behavioral groupings can then be compared against the typical ranges in Tables 3-5 to determine how well the score profile for the sample (dog) fits the various cluster profiles. This determination can then be tested against all 4 clusters (or all clusters that are not excluded), particularly in instances where the data/scores related fairly well to multiple clusters in order to find the best fit. Alternatively, in the majority of instances where a specific principal component behavioral group and associated behavioral signs fit very well within the typical score profile of one cluster and no other, then the labeling or categorization into that specific cluster can be reasonably assured. If the fit is not very close to the closest cluster, then determination of a fit within a given behavioral cluster can be determined based on the best match. If there is still not a good match to one of the clusters, further factors may be considered for each behavioral profile, such as i) for how many behavioral groupings is there an incomplete match at the level of typical score? ii) for how many principal component behavioral groupings is there an incomplete match at the level of the range should the behavioral sign be present? (if the sign must be present then this will be the same as the first point here); iii) if a behavioral sign is present in a principal component behavioral group, the likelihood to be in that group lies on what percentage of that cluster show “1 or more behavioral signs” of that behavioral grouping; and/or iv) How far out is the behavioral grouping score? With respect to iii), if a dog shows no behavioral signs of exit frustration (Ef) and in the end the dog is matched to Cluster B and Cluster D, e.g., 2 Yes for B and 2 Yes for D, the dog would most likely be classified in Cluster D, since 76% of this group shows 0 behavioral signs while only 28% of group B shows no behavioral signs for exit frustration (Ef). Other similar evaluations can occur as may be appropriate for similar situations. At some point, if it is unclear as to the closest match, there may be a reason for interjecting a “professional judgement” into the algorithm to arrive at the closest match. However, as the machine-learning algorithm gets smarter by introduction of additional data, e.g., by new data entered by end users, the use of professional judgment may become rarer over time.

In accordance with the present disclosure, an SRP identification framework or workflow (including quality control assessments) can be established with respect to separation-related problems (SRP) in pets, such as when a dog is left alone. The identification framework can be based on SRP dataset(s) that utilizes behavioral signs and principal component behavioral groupings, such as those shown and described with reference to Table 1 for dogs as an example, e.g., 54 behavioral signs and 7 principal component behavioral groupings. The 54 behavioral signs (or any number of behavioral signs that may be appropriate for a given type of pet or other system for use with dogs), can be gathered by inquiring of a pet caregiver, such as a pet owner, a veterinarian, kennel personnel, etc. The inquiry can be in the form of a questionnaire about the pet which can be correlated to scoring the behavioral signs using binary annotation or code, for example. There may be fewer or more questions than the number of behavioral signs, e.g., 54 behavioral signs, as it may take fewer or more questions to accurately score each of the behavioral signs of the pet. Questions can be generated by behaviorists or other competent professionals to provide reasonably accurate scoring for each of the behavioral signs.

By way of example, the identification framework and related SRP dataset(s) can be used to categorize pets, e.g., dogs, into one of several clusters, such as the four (4) behavioral clusters shown by way of example in Table 2. The systems and methods described herein can be established using machine-learning algorithms based on a dataset of several hundred (or more) dogs as the sample size for purposes of training the artificial intelligence, e.g., at least about 250 dogs, from about 400 to about 2,500 dogs, or from about 500 to about 1,000 dogs.

Upon training the machine-learning algorithm, each dog (or sample) can be appropriately labeled or categorized via a binary vector length annotated by one (or more) of the behavioral clusters, e.g., Cluster A, Cluster B, Cluster C, or Cluster D. The machine-learning algorithm can thus use the 54 binary vectors (or behavioral signs) to predict its appropriate behavioral cluster, which can be predicted as a result of a machine-learning algorithm that has been adequately trained with training data with a sufficiently large sample size, e.g., at least 250 dogs (or other type of pet).

In accordance with examples herein, computer programs and/or applications can be implemented to provide pet caregivers, e.g., owner, veterinarian, trainer, kennel service provider, etc., with tools or workflows for identifying SRPs and/or treating SRPs in a pet(s). Though the data presented herein relates more particularly to dogs, this type of system can likewise be implemented with other pets, such as cats, rats, ferrets, hamsters, rabbits, birds, reptiles, e.g., iguanas, pigs, horses, goats, cows, etc. In examples herein, the computer programs and/or applications benefit from machine-learning, e.g., artificial intelligence (AI), using any of a number of machine-learning protocols. For example, solution development can include the use of various protocols and the collection of datasets for training a machine-learning algorithm for generating machine-learning solutions suitable for providing web-based services, e.g., use of Python, FLASK library, REST API, or the like. In such an example, an algorithm(s) can be used to convert binary features into numbers and predict appropriate labels, e.g., related to dog population clusters, based on the highest probability generated from a training data set.

A machine-learning algorithm can be used based on any of a number of approaches, such as a metric learning approach. Regarding the metric learning approach, the algorithm can be enabled to treat binary features as a binary number and convert individual sets of features to a number. With this data, the algorithm can then predict which dog population cluster (See Table 2 above) would have the highest probability of being accurate with respect to a specific pet based on the training data. If there is not a close match as presented by the training data, the algorithm can return with the closest source feature represented number corresponding with a dog population cluster.

To provide an example of training data that can be collected and inputted for purposes of machine-learning, a dataset of 589 dogs (sample n=589) were sub-categorized or annotated with one of four labels representing one of four population clusters corresponding to four types of SRP in dogs. The population clusters were established in accordance with that shown and described in Tables 1-5 previously. The population clusters were as follows: Cluster A=82 dogs, Cluster B=128 dogs, Cluster C=259 dogs, and Cluster D=118 dogs. Each dog was represented by a binary vector of length 54 (corresponding with the 54 behavioral signs) and each dog was also annotated by one of the behavioral cluster labels (A-D). With the training data in place, a machine-learning algorithm may be given a binary vector of 54 in size to accurately predict behavioral clusters (or labels) going forward.

In this example, Python (Version 3.7.5) was utilized as a standard programming language for the machine-learning based solution, and the FLASK library was used for the HTTP server wrapper to provide the HTTP server interface. FLASK is a microweb framework written in Python that does not require particular tools or libraries with no database abstraction layer, form validation, or other components with pre-existing third-party libraries providing common functions. FLASK does support extensions that can add application features as if implemented, with the extensions existing for object-relational mappers, form validation, upload handling, various open authentication technologies, and other framework related tools.

In this example, the application programming interface (API) was established with two endpoints: “/” [GET] for querying the model and “/tat” [GET] for retraining the model in this example. The code was then formatted following industry standards defined in PEP8 and further enforced by the “black” coding standard to allow review and maintenance. PEP8 establishes the best practices regarding the writing of Python code, and the black coding standards are typically implemented to establish consistency, generality, readability, reducing git diffs (a command to show the difference between files in two commits or between current repository and previous commit), etc.

In accordance with a specific example of implementing machine-learning using these tools, three files can be used, namely by example:
i) a “main.py” file as a single entry point for the model training and a REST API wrapper for which it can be implemented using FLASK;
ii) a “algo.py” file as the main logic of the project; and
iii) a “consts.py” file with a set of constants used in the project at hand.

The algorithm was based on the source features or dataset that was collected and were all based on binary information. In other words, each set of behavioral signs could be converted to a number for each of the principal component behavioral groupings, and the behavioral cluster label with the highest probability when compared against the training data could be returned accordingly. Thus, after training the dataset with several hundred samples, dogs may then be evaluated using the artificial intelligence based on the training dataset and any other dogs subsequently presented to the machine-learning algorithm may be evaluated (which may further solidify the training data with additional datapoints to consider). The cluster assigned can be based on the closest source features or behaviors represented numerically for that particular pet, e.g., dog.

In some examples, in making the appropriate behavioral cluster determination for a dog, the machine-learning can be based on a metric learning approach, for example. Metric learning can be based on the distance between two data points. For example, standard distances used for metric learning can be Euclidean, City-Block, Cosine, etc., to name a few, using prior knowledge of the domain. Distance metric learning aims at automatically constructing task-specific distance metrics from (weakly) supervised data, in a machine-learning manner. The learned distance metric can then be used to perform various tasks, such as k-NN classification, clustering, information retrieval, pattern matching, etc.

In the context of the present disclosure regarding metric learning, the algorithm can be enabled to treat binary features as a binary number and convert individual sets of features to a number. With this data, the algorithm can then predict which dog population cluster (See Table 2 above) would have the highest probability of being accurate with respect to a specific pet based on the training data. If there is not a close match as presented by the training data, the algorithm can return with the closest source feature represented number corresponding with a dog population cluster. Metric learning can be based on supervised learning or weakly supervised learning, for example. Examples of machine-learning algorithms used for metric learning can be based on any of a number of systems, including those using decision trees (e.g. random forests), k-nearest neighbors, support vector machines (SVM), neural networks (NN), recurrent neural networks (RNN), convolutional neural networks (CNN), probabilistic neural networks (PNN), heuristics, regression, light gradient-boosting machine (GBM), or the like.

An example metric learning system as used herein may be based on a machine-learning model, such as TPOT. For example, TPOT for automated machine-learning (AutoML) in Python relates to automatically discovering well-performing models for predictive modeling tasks and/or for optimizing machine-learning pipelines, which can be configured for implication with very little user involvement. Thus, the AI can be trained by using TPOT to assist in finding the best or even optimal pipeline for tasks based on a stochastic gradient decent algorithm. In some examples, TPOT can make use of the Scikit-Learn machine-learning library for data transforms and machine-learning algorithms and/or can use genetic programming stochastic global search procedures to efficiently discover a top-performing model pipeline for a given dataset. TPOT can be beneficial for use as it can automate some of the more tedious portions of the machine-learning by intelligently exploring thousands of possibilities (or pipelines) to find the best fits for the data that has been entered. After entering raw data (and cleaning the data if needed), a TPOT automated machine-learning pipeline may be initiated leading to model selection and/or parameter optimization, for example. The automated TPOT process may consider and/or carry out any of a number of actions, such as feature selection, preprocessing, construction, etc., to name a few. In this instance, the TPOT machine-learning pipeline can be programmed to categorize dogs into behavioral clusters based on the behavioral signs and/or principal component behavioral groupings described in detail in Tables 1-5, for example. Once TPOT is finished with its processing or other tasks (or when it gets far enough along to provide meaningful clustering), the TPOT can be configured to provide the best pipeline or pipeline options.

In some examples, TPOT can use a tree-based pipeline optimization tool, and in some examples, can validate using model validation software, e.g., k-fold cross-validation, to gain robustness. Essentially, the dataset collected as described previously can be split into a training set (from which a proposed algorithm is derived) and a validation set (from which the proposed algorithm is applied and tested). In further detail, k-fold cross-validation methods can implement the splitting of a dataset into k equal-sized and distinct subsets or folds. For example, cross-validation can be carried out by repeatedly splitting the study population into training groups and validation groups multiple times in different ways in order to achieve a more statistically resilient evaluation of the performance of the prediction tool or workflow. More specifically, this type of model can be trained on k−1 folds and tested on the remaining fold(s), for example. This method can be repeated “k” times, with each fold serving as a validation set, e.g., once. By averaging the performance metrics obtained from each fold, such as accuracy, F1 score, recall, precision, etc., a more statistically resilient evaluation of the model's abilities and a more accurate model can be obtained. For example, this system can assist with estimating model performance on unseen data and assess its ability to generalize well. Once the tree-based pipeline optimization tool has run or has sufficiently completed to obtain meaningful clustering (and has been cross-validated in some examples), the best model can then be linearly fitted using the stochastic gradient descent (SGD) algorithm to obtain the final model for the solution. SGD is an iterative method for optimizing an objective function with suitable smoothness properties, e.g. differentiable or subdifferentiable. It may be considered to be a stochastic approximation of gradient descent optimization, since it can replace an actual gradient (calculated from the data set).

In further detail regarding k-fold cross-validation, this type of validation can be used to evaluate the accuracy of the dog population clustering and/or any other labeling that may be applicable. In further detail, the k-fold cross-validation can be used to evaluate the performance and generalization ability of the machine-learning model. For example, the k-fold cross-validation method can implement the splitting of a dataset into k equal-sized and distinct subsets or folds. This type of model can be trained on k−1 folds and tested on the remaining fold(s). This method can be repeated “k” times, with each fold serving as a validation set, e.g., once. By averaging the performance metrics obtained from each fold, such as accuracy, F1 score, recall, precision, etc., a more statistically resilient evaluation of the model's abilities and a more accurate model can be obtained. For example, this system can assist with estimating model performance on unseen data and assess its ability to generalize well.

To continue with the example that utilized 589 samples (dogs) to train and validate the machine-learning algorithm, the 589 cohort of dogs was split into a training cohort (from which the algorithm can be derived) and a validation cohort (from which the algorithm can be applied and tested) to develop a machine-learning-based prediction tool or workflow. Cross-validation was carried out by repeatedly splitting the study population into training and validation groups multiple times in different ways. This approach can be used to achieve a more statistically resilient evaluation of the performance of the prediction tool or workflow. In particular, the k-fold cross-validation method, which splits the data into k identical-sized and pairwise distinct cohorts, was used in this study. Each of these two cohorts was used as the validation cohort once, and the remaining cohorts were used as training cohorts, resulting in “k” validations. The average performance, computed for four different metrics to obtain a clear picture of the model's abilities, of the prediction tool or workflow in these validation cohorts was computed to estimate the performance of the prediction tool. Machine-learning/training along with the k-fold validation (or other validation tool), for example, can provide an accuracy of at least about 0.8 (or 80%), at least about 0.85 (or 85%), or at least about 0.9 (or 90%), which are accurate enough for effective use.

Table 6 summarizes specific data based on the 589 dogs used in training and validating the artificial intelligence, where 5-folds were obtained for the testing cohorts with an average value presented in the last row. As can be seen, the overall accuracy of the model was calculated to be 0.929, or 92.9% accurate. This outcome shows the model is well generalized over all four behavioral clusters.

TABLE 6
Summary of Model Performance (divided into 5 k-folds)

| Fold | Accuracy | F1 | Recall | Precision |
|---|---|---|---|---|
| 1 | 0.907 | 0.904 | 0.907 | 0.917 |
| 2 | 0.983 | 0.983 | 0.983 | 0.983 |
| 3 | 0.924 | 0.925 | 0.924 | 0.926 |
| 4 | 0.906 | 0.905 | 0.905 | 0.905 |
| 5 | 0.923 | 0.925 | 0.923 | 0.927 |
| Average | 0.929 | 0.928 | 0.929 | 0.927 |

Deployment of the machine-learning algorithms of the present disclosure can include the use of computer program or application protocols (over a cloud-based network or otherwise), and/or may include cloning of a repository cloning, installing program requirements, running the project from a main file, e.g., main.py file if using Python, etc. In some examples, the use of a “nohup” command can be implemented to prevent the program from being terminated if the terminal is closed. Any of a number of servers can be used to carry out the AI, such as Linux servers, Microsoft servers, Amazon servers, etc. In some examples, local servers or client devices can be used in whole or in part, or more typically, cloud-based solutions may be used (or partially used) for one or more of data input storage, cloud computing, data analysis, etc. Example cloud-based platforms suitable for use include Google Cloud Platform (AppEngine), Microsoft Azure, Amazon Web Services, Alibaba Cloud, IBM Red Hat, HPC Cloud Services, Dell VMware, Cisco ACI, etc.

As an example, in order to deploy the machine-learning solution using a Linux server, the following steps may be useful, namely:
clone the repository into the server;
install the ‘requirements.txt’ file (if PIP solution installed, “pip install-r requirements.txt” from the console); and
run the project from the main.py file (either by “python main.py” or “python3 main.py”, depending on the version on the server).
   In some instances, if the terminal from SSH is not monitored and the app is not run from the root user, the program may be terminated. Thus, the use of the “nohup” command can be included to prevent this from happening. In further detail, as the machine-learning protocol is “state-less,” the use of the Google Cloud Platform and the AppEngine solution may be suitable for use.

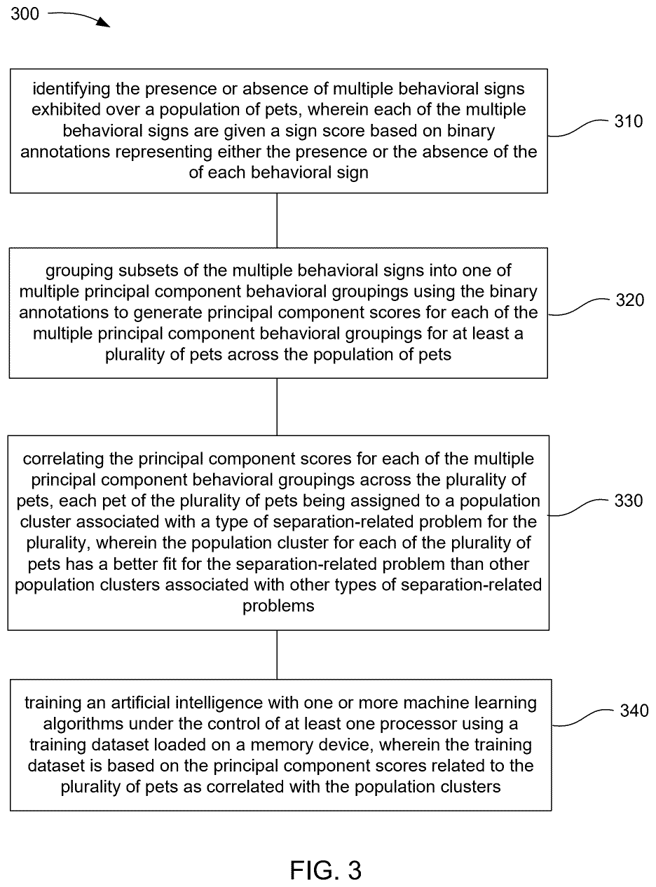

In accordance with the examples set forth herein, FIG. 3 illustrates a flow diagram which sets forth a method 300 of generating a machine-learning workflow for identifying separation-related problems in a pet. This method can include identifying 310 the presence or absence of multiple behavioral signs exhibited over a population of pets. Each of the multiple behavioral signs can be given a sign score based on binary annotations representing either the presence or the absence of each the behavioral signs. The method can further include grouping 320 subsets of the multiple behavioral signs into one of multiple principal component behavioral groupings using the binary annotations to generate principal component scores for each of the multiple principal component behavioral groupings for at least a plurality of pets across the population of pets, and correlating 330 the principal component scores for each of the multiple principal component behavioral groupings across the plurality of pets. For example, each pet of the plurality of pets can be assigned to a population cluster associated with a type of separation-related problem for the plurality of pets. The population cluster for each of the plurality of pets may have a better fit for the separation-related problem than other population clusters associated with other types of separation-related problems. In some examples, the method can also include training an artificial intelligence with one or more machine-learning algorithms under the control of at least one processor using a training dataset loaded on a memory device, wherein the training dataset is based on the principal component scores related to the plurality of pets as correlated with the population clusters. With respect to the methods (and systems/machine-readable storage media utilizing these methods), training the artificial intelligence can include finding or optimizing a machine-learning pipeline. For example, finding or optimizing the machine-learning pipeline may result in the use of a linearly fitted model utilizing a stochastic gradient decent algorithm, or some other similar algorithm.

In some examples, the training dataset can be validated using a validation dataset collected using a second population of pets (with data collected in the same manner as the statistical analysis used to generate the training dataset). The second population of pets can be used to validate the artificial intelligence by applying and testing the one or more machine-learning algorithms, e.g., using tree-based pipeline optimization, k-fold cross-validation, or a combination thereof.

In some examples, when collecting data for inclusion in the training dataset or the validation dataset, a portion or all of the binary annotations for at least some of the behavioral signs are scored 0 for the absence of the behavioral signs and 1 for the presence of the behavioral signs, and/or a portion of the binary annotations for at least one of the behavioral signs is scored −1 for the presence of a positive behavioral sign and 1 for a negative behavioral sign. In some examples, at least 50 behavioral signs may be considered for the grouping of subsets of the multiple behavioral signs into one of multiple principal component behavioral groupings. In other examples, identifying the presence or absence of multiple behavioral signs can be carried out by a pet caregiver answering inquiries about the pet using a client device connected to a computer network in communication with the one or more machine-learning algorithms. The principal component behavioral groupings can be used for categorization into at least four population clusters that are each associated with a unique separation-related problem. Correlating the principal component scores with the population cluster can include exclusion of at least one behavioral cluster as a possibility in some examples, and/or in other examples, correlating the principal component scores with a population cluster can include assigning a closest source feature represented by the principal component scores of the multiple principal component behavioral groupings using metric learning.

Using a dog as a specific example, the principal component behavioral groupings can include one or more, two or more, or three or more, or all of the groupings selected from exit frustration (Ef), redirected frustration (Rf), social panic (Sp), elimination (E), reactive communication (Rc), immediate frustration (If), or noise sensitivity (Ns). With dogs, example population clusters can include one or more, two or more, or three or more, or all of the population clusters selected from exit frustration, redirected reactive, reactive inhibited, or boredom.

In accordance with examples herein, it is noted that the use of any processor(s) and/or memory(s) may be located onboard or located remotely relative to any client device, such as a computer, a tablet, a smartphone, etc., that may be used by a pet caregiver, e.g., owner, veterinarian, trainer, kennel service provider, etc. Furthermore, it is noted that the features and advantages described herein are not all-inclusive and, in particular, many additional features with related advantages may be implemented in connection with the technology described herein. Moreover, it should be noted that the language used in the specification has been principally selected for readability and instructional purposes, and not to limit the scope of the inventive subject matter.

With respect to the systems, methods, non-transitory machine-readable storage medium, etc., these related technologies can be used in the context of other connected computers and/or server systems by wireless or wired connection. For example, the methods, systems, storage media, etc., can include the use of client devices and/or analysis server systems, one or both of which can be in communication via network. In some aspects, the analysis server systems may be implemented using a single server or a plurality of servers. For example, a client device can be interactive with and implemented utilizing the analysis server systems. In more particular detail regarding client devices, these may include, for example, desktop computers, laptop computers, smartphones, tablets, and/or any other user interface suitable for communicating with a network where one or more machine-learning algorithms are located. Client devices can obtain a variety of data as entered by the pet caregiver, which then may use artificial intelligence or machine-learning to identify a separation-related problem (SRP), and in some instances, recommend treatment for a pet, such as a dog. In other words, the inputted information and AI generated identification of the SRP(s) and/or treatment recommendation can provide data and insights regarding the pet via one or more computer programs or software applications, and/or provide data and/or insights to the analysis server systems. The computer programs or software applications can provide data characterizing the SRP that the pet may be suffering from and/or provide some health information related to the condition. In some examples, the software applications may obtain data from the analysis server systems for processing and/or display.

In some examples, the network to which the client device(s) connect may include an analysis server system for collecting data inputted by the pet caregiver and running the artificial intelligence algorithms based on that data, which can be used to assist the artificial intelligence in its learning as established from training data. The training data, for example, can be based on multiple independent analyses conducted by multiple pet behaviorist, for example. The analysis server systems can provide data and insights regarding one or more pets and or transmit data and/or insights to the client devices. In a number of examples, the analysis server system(s) can obtain data from multiple client devices, identify cohorts of pets or a specific pet identified within the obtained data based on one or more characteristics of the pet(s), and determine insights for the cohorts of pets. These insights for a cohort of pets can be used to properly identify SRPs, and in some instances, provide recommendations for a particular pet that has characteristics in common with the characteristics of the cohort. In some examples, the analysis server systems can provide a portal, e.g., a web site or an application, for veterinarians or other pet care professionals to access information regarding a particular pet.

In some examples, the software application can download data to the client device to operate without connection with the network. This would enable the user to operate the application locally as a non-network client device without sending data through the network at the time of use. For clarity, the term “non-network” client device does not infer it is not also connected via the cloud or other network, but merely that the use of the network is not needed to operate the software application.

Any of the computing devices described herein, including the client devices, analysis server systems, non-networked client devices, etc., can include a single computing device, multiple computing devices, a cluster of computing devices, or the like. A computing device can include one or more physical processors communicatively coupled to one or more memory devices, input/output devices, or the like. As used herein, a processor may also be referred to as a central processing unit (CPU).

Additionally, as used herein, a processor can include one or more devices capable of executing instructions encoding arithmetic, logical, and/or I/O operations. In one illustrative example, a processor may implement a Von Neumann architectural model and may include an arithmetic logic unit (ALU), a control unit, and/or a plurality of registers. In some aspects, a processor may be a single core processor that is typically capable of executing one instruction at a time (or process a single pipeline of instructions) and/or a multi-core processor that may simultaneously execute multiple instructions. In some examples, a processor may be implemented as a single integrated circuit, two or more integrated circuits, and/or may be a component of a multi-chip module in which individual microprocessor dies are included in a single integrated circuit package and hence share a single socket. As described herein, a memory refers to a volatile or non-volatile memory device, such as RAM, ROM, EEPROM, or any other device capable of storing data. Input/output devices can include a network device (e.g., a network adapter or any other component that connects a computer to a computer network), a peripheral component interconnect (PCI) device, storage devices, disk drives, sound or video adaptors, photo/video cameras, printer devices, keyboards, displays, etc. In some aspects, a computing device provides an interface, such as an API or web service, which provides some or all of the data to other computing devices for further processing. Access to the interface can be open and/or secured using any of a variety of techniques, such as by using client authorization keys, as appropriate to the requirements of specific applications of the disclosure.

The network(s) herein can include, for example, a LAN (local area network), a WAN (wide area network), telephone network (e.g., Public Switched Telephone Network (PSTN)), Session Initiation Protocol (SIP) network, wireless network, point-to-point network, star network, token ring network, hub network, wireless networks (including protocols such as EDGE, 3G, 4G LTE, Wi-Fi, 5G, WiMAX, and the like), the Internet, or the like. A variety of authorization and authentication techniques, such as username/password, Open Authorization (OAuth), Kerberos, SecureID, digital certificates, and more, may be used to secure the communications. It will be appreciated that network connections described herein are illustrative, and any means of establishing one or more communication links between the computing devices may be used.

In further detail, the computer programs or software applications described herein can include caregiver notifications. For example, information regarding the pet can be transmitted, which may include notification related to the pet's behaviors, and furthermore, can be generated based on the inputted questionnaire and the artificial intelligence-generated identification of SRP(s) and/or recommendations for the pet. These features can work together to identify or represent any of a number of categorized events, historical events, noted behaviors, daily behaviors, occasional behaviors, etc.

Regarding operation of the computer programs or software applications described herein, a variety of user interfaces can be provided to ensure the proper installation, configuration, and usage. These user interfaces can provide instruction to users, solicit information from users, and/or provide insights into the behaviors and potential concerns with one or more pets.

In some examples, user interfaces for establishing a pet profile may be used. Examples of user interfaces for establishing a pet profile include, a user interface of a start screen for a pet profile establishment process, a user interface of an introductory screen for a pet profile establishment, a user interface for entering a pet's name, a user interface for entering a pet's physical characteristics, e.g., sex, reproductive status, current body condition, a user interface for examining specific pet body parts or size dimensions at various locations, a user interface of an ending screen for a pet profile establishment process, etc. In some examples, user interfaces for expert or professional advice notifications may be used.

It is noted that although the systems, methods, and software applications described herein include various example details, methods may be performed by various software and/or hardware configurations, including various processing logic that may include hardware (circuitry, dedicated logic, etc.), software, or a combination of both. For example, methods may be implemented and executed as instructed on a machine, where the instructions are included on at least one computer readable medium or one non-transitory machine-readable storage medium.

It will be appreciated that all of the disclosed methods and procedures described herein can be implemented using one or more computer programs, components, applications, program modules, etc. These components may be provided as a series of computer instructions on any conventional computer readable medium or machine-readable medium, including volatile or non-volatile memory, such as RAM, ROM, flash memory, magnetic or optical disks, optical memory, or other storage media. The instructions may be provided as software or firmware and/or may be implemented in whole or in part in hardware components such as ASICs, FPGAs, DSPs, or any other similar devices. The instructions may be configured to be executed by one or more processors which, when executing the series of computer instructions, performs or facilitates the performance of all or part of the disclosed methods and procedures. As will be appreciated, the functionality of the program modules may be combined or distributed as desired in various aspects of the disclosure.

### EXAMPLES

In accordance with the disclosure herein, the following examples are illustrative of several embodiments of the present technology.

1. A method of generating a machine-learning workflow for identifying separation-related problems in a pet, comprising:
identifying the presence or absence of multiple behavioral signs exhibited over a population of pets, wherein each of the multiple behavioral signs are given a sign score based on binary annotations representing either the presence or the absence of each of the behavioral signs;
grouping subsets of the multiple behavioral signs into one of multiple principal component behavioral groupings using the binary annotations to generate principal component scores for each of the multiple principal component behavioral groupings for at least a plurality of pets across the population of pets;
correlating the principal component scores for each of the multiple principal component behavioral groupings across the plurality of pets, each pet of the plurality of pets being assigned to a population cluster associated with a type of separation-related problem for the plurality, wherein the population cluster for each of the plurality of pets has a better fit for the separation-related problem than other population clusters associated with other types of separation-related problems; and
training an artificial intelligence with one or more machine-learning algorithms under the control of at least one processor using a training dataset loaded on a memory device, wherein the training dataset is based on the principal component scores related to the plurality of pets as correlated with the population clusters.

2. The method of example 1, wherein training the artificial intelligence includes finding or optimizing a machine-learning pipeline.

3. The method of one or more of examples 1 or 2, further comprising finding or optimizing the machine-learning pipeline results in the use of a linearly fitted model utilizing a stochastic gradient decent algorithm.

4. The method of one or more of examples 1 to 3, further comprising:
generating a validation dataset based on a second populations of pets; and
validating the artificial intelligence by applying and testing the one or more machine-learning algorithms using the validation dataset.

5. The method of example 4, wherein validating the artificial intelligence includes using tree-based pipeline optimization, k-fold cross-validation, or a combination thereof.

6. The method of one or more of examples 1 to 5, wherein a portion or all of the binary annotations for at least some of the behavioral signs are scored 0 for the absence of the behavioral signs and 1 for the presence of the behavioral signs.

7. The method of example 6, wherein a portion of the binary annotations for at least one of the behavioral signs is scored −1 for the presence of a positive behavioral sign and 1 for a negative behavioral sign.

8. The method of one or more of examples 1 to 7, wherein at least 50 behavioral signs are considered for the grouping of subsets of the multiple behavioral signs into the one of multiple principal component behavioral groupings.

9. The method of one or more of examples 1 to 8, wherein identifying the presence or absence of multiple behavioral signs is carried out by a pet caregiver answering inquiries about the pet using a client device connected to a computer network in communication with the one or more machine-learning algorithms.

10. The method of one or more of examples 1 to 9, wherein the pet is a dog, and the principal component behavioral groupings include one or more of exit frustration (Ef), redirected frustration (Rf), social panic (Sp), elimination (E), reactive communication (Rc), immediate frustration (If), or noise sensitivity (Ns).

11. The method of one or more of examples 1 to 10, having at least four population clusters that are each associated with a unique separation-related problem.

12. The method of one or more of examples 1 to 11, wherein the pet is a dog, and the population clusters include one or more of exit frustration, redirected reactive, reactive inhibited, or boredom.

13. The method of one or more of examples 1 to 12, wherein correlating the principal component scores with the population cluster includes exclusion of at least one behavioral cluster as a possibility.

14. The method of one or more of examples 1 to 13, wherein correlating the principal component scores with a population cluster includes assigning a closest source feature represented by the principal component scores of the multiple principal component behavioral groupings using metric learning.

15. A machine-learning system for identifying separation-related problems in a pet, comprising:
at least one processor and at least one memory device including a data store to receive behavioral information related to an individual pet which is entered into a client device to correlate the behavioral information with a plurality of binary sign scores related to the presence or absence of multiple behavioral signs related to the individual pet,
group subsets of the multiple behavioral signs into one of multiple principal component behavioral groupings to generate principal component scores for each of the multiple principal component behavioral groupings, and
correlating the multiple principal component scores for each of the multiple principal component behavioral groupings to assign the individual pet to a population cluster associated with a type of separation-related problem, wherein the population cluster has a better fit for the separation-related problem than other population clusters associated with other types of separation-related problems.

16. The machine-learning system of example 15, wherein the data store further includes instructions that, when executed, cause the system to report the population cluster, the separation-related problem, or both to the pet caregiver at the client device.

17. The machine-learning system of one or more of examples 15 or 16, wherein at least a portion of the instructions are generated using a training dataset, wherein the training dataset is derived from a population of pets of the same species as the individual pet.

18. The machine-learning system of example 17, wherein the training dataset is used to find or optimize a machine-learning pipeline which returns accurate identification of the population cluster for the individual pet with at least 90% confidence.

19. The machine-learning system of example 18, wherein the machine-learning pipeline includes a linearly fitted model utilizing tree-based pipeline optimization resulting in the use of a stochastic gradient decent algorithm.

20. The machine-learning system of example 18, wherein the instructions are validated using a validation dataset derived from a second population of pets of the same species as the individual pet, wherein the validation dataset confirms the accuracy of the identification of the population cluster for the individual pet.

21. The machine-learning system of example 20, wherein the instructions are validated using tree-based pipeline optimization and k-fold cross-validation.

22. The machine-learning system of one or more of examples 15 to 21, wherein the pet is a dog, and the principal component behavioral groupings include at least one of exit frustration (Ef), redirected frustration (Rf), social panic (Sp), elimination (E), reactive communication (Rc), immediate frustration (If), or noise sensitivity (Ns); and wherein the population clusters are each associated with a unique separation-related problem including at least one of exit frustration, redirected reactive, reactive inhibited, or boredom.

23. A non-transitory machine-readable storage medium having instructions embodied thereon, the instructions when executed by one or more processors, cause the one or more processors to perform to:
correlate the behavioral information with a plurality of binary sign scores related to the presence or absence of multiple behavioral signs related to the individual pet and group subsets of the multiple behavioral signs into one of multiple principal component behavioral groupings to generate principal component scores for each of the multiple principal component behavioral groupings,
correlate the multiple principal component scores for each of the multiple principal component behavioral groupings to assign the individual pet to a population cluster associated with a type of separation-related problem, wherein the population cluster has a better fit for the separation-related problem than other population clusters associated with other types of separation-related problems, and
report the population cluster, the separation-related problem, or both to the pet caregiver at the client device.

24. The non-transitory machine-readable storage medium of example 23, wherein at least a portion of the instructions are generated by machine-learning using a training dataset derived from a population of pets of the same species as the individual pet, and wherein the instructions are validated using a validation dataset derived from a second population of pets of the same species as the individual pet.

25. The non-transitory machine-readable storage medium of one or more of examples 23 or 24, wherein the pet is a dog, and wherein the principal component behavioral groupings include exit frustration (Ef), redirected frustration (Rf), social panic (Sp), elimination (E), reactive communication (Rc), immediate frustration (If), noise sensitivity (Ns), or a combination thereof, and wherein population clusters are each associated with a unique separation-related problem selected from exit frustration, redirected reactive, reactive inhibited, boredom, or a combination thereof.

### Definitions

As used herein, “about,” “approximately” and “substantially” are understood to refer to numbers in a range of numerals, for example the range of −10% to +10% of the referenced number, −5% to +5% of the referenced number, −1% to +1% of the referenced number, or −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Thus, a disclosure of an embodiment using the term “comprising” includes a disclosure of embodiments “consisting essentially of” and “consisting of” the components identified.

The term “and/or” used in the context of “X and/or Y” should be interpreted as “X,” or “Y,” or “X and Y.” Similarly, “at least one of X or Y” should be interpreted as “X,” or “Y,” or “X and Y.”

The terms “pet” and “animal” are used synonymously herein and mean any pet which can benefit from the machine-learning algorithm and implementation thereof in accordance with the present disclosure, with non-limiting examples including a dog, a cat, a rat, a ferret, a hamster, a rabbit, an iguana, a pig, a bird, etc. The data presented herein is based on a dog study and machine-learning for dogs suffering from separation-related problems (SRPs), but the pet can be any suitable domestic animal where data is collected for training an artificial intelligence or machine-learning algorithm.

As used herein, ranges are in shorthand so as to avoid having to list and describe each and every value within the range. Any appropriate value within the range can be selected, where appropriate, as the upper value, lower value, or the terminus of the range, and thus should be interpreted flexibly to include the numerical values explicitly recited as the limits of the range, and also to include individual numerical values or sub-ranges encompassed within that range as if numerical values and sub-ranges are explicitly recited. As an illustration, a numerical range of “about 1% to about 5%” should be interpreted to include the explicitly recited values of about 1% to about 5%, and also to include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting one numerical value. Furthermore, such an interpretation should apply regardless of the breadth of the range or the characteristics being described.

The term “example(s)” or “embodiment(s),” particularly when followed by a listing of terms, is merely exemplary and illustrative and should not be deemed to be exclusive or comprehensive. The terms “example” or “embodiment” or the use of the phrase “such as” is considered to be merely exemplary and illustrative and should not be deemed to be exclusive or comprehensive.

The systems, methods, computer programs or software applications, non-transitory storage media, etc., disclosed herein are not limited to particular methodology, protocols, etc., that are described in detail herein, as implementation details may vary within the scope of the present disclosure. Further, the terminology used herein is for the purpose of describing particular embodiments only, and is not intended to and does not limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, certain compositions, methods, articles of manufacture, or other means or materials are described herein.

As used herein, a plurality of elements, compositional components, and/or materials may be presented in a common list for convenience. However, these lists should be construed as though individual members of the list are individually identified as separate and unique members. Thus, no individual member of such list should be construed as a de facto equivalent of any other member of the same list solely based on presentation in a common group without indications to the contrary.

What is claimed is:

1. A method of generating a machine-learning workflow for identifying separation-related problems in a pet, comprising:
identifying the presence or absence of multiple behavioral signs exhibited over a population of pets, wherein each of the multiple behavioral signs are given a sign score based on binary annotations representing either the presence or the absence of each of the behavioral signs;
grouping subsets of the multiple behavioral signs into one of multiple principal component behavioral groupings using the binary annotations to generate principal component scores for each of the multiple principal component behavioral groupings for at least a plurality of pets across the population of pets;
correlating the principal component scores for each of the multiple principal component behavioral groupings across the plurality of pets, each pet of the plurality of pets being assigned to a population cluster associated with a type of separation-related problem for the plurality, wherein the population cluster for each of the plurality of pets has a better fit for the separation-related problem than other population clusters associated with other types of separation-related problems; and
training an artificial intelligence with one or more machine-learning algorithms under the control of at least one processor using a training dataset loaded on a memory device, wherein the training dataset is based on the principal component scores related to the plurality of pets as correlated with the population clusters.

2. The method of claim 1, wherein training the artificial intelligence includes finding or optimizing a machine-learning pipeline.

3. The method of claim 2, further comprising finding or optimizing the machine-learning pipeline results in the use of a linearly fitted model utilizing a stochastic gradient decent algorithm.

4. The method of claim 1, further comprising:
generating a validation dataset based on a second populations of pets; and
validating the artificial intelligence by applying and testing the one or more machine-learning algorithms using the validation dataset.

5. The method of claim 4, wherein validating the artificial intelligence includes using tree-based pipeline optimization, k-fold cross-validation, or a combination thereof.

6. The method of claim 1, wherein a portion or all of the binary annotations for at least some of the behavioral signs are scored 0 for the absence of the behavioral signs and 1 for the presence of the behavioral signs.

7. The method of claim 6, wherein a portion of the binary annotations for at least one of the behavioral signs is scored −1 for the presence of a positive behavioral sign and 1 for the presence of a negative behavioral sign.

8. The method of claim 1, wherein at least 50 behavioral signs are considered for the grouping of subsets of the multiple behavioral signs into the one of multiple principal component behavioral groupings.

9. The method of claim 1, wherein identifying the presence or absence of multiple behavioral signs is carried out by a pet caregiver answering inquiries about the pet using a client device connected to a computer network in communication with the one or more machine-learning algorithms.

10. The method of claim 1, wherein the pet is a dog, and the principal component behavioral groupings include one or more of exit frustration (Ef), redirected frustration (Rf), social panic (Sp), elimination (E), reactive communication (Rc), immediate frustration (If), or noise sensitivity (Ns).

11. The method of claim 1, having at least four population clusters that are each associated with a unique separation-related problem.

12. The method of claim 1, wherein the pet is a dog, and the population clusters include one or more of exit frustration, redirected reactive, reactive inhibited, or boredom.

13. The method of claim 1, wherein correlating the principal component scores with the population cluster includes exclusion of at least one behavioral cluster as a possibility.

14. The method of claim 1, wherein correlating the principal component scores with a population cluster includes assigning a closest source feature represented by the principal component scores of the multiple principal component behavioral groupings using metric learning.

15. A machine-learning system for identifying separation-related problems in a pet, comprising at least one processor and at least one memory device including a data store to receive behavioral information related to an individual pet which is entered into a client device by a pet caregiver, wherein the data store also includes instructions that, when executed, cause the system to:
correlate the behavioral information with a plurality of binary sign scores related to the presence or absence of multiple behavioral signs related to the individual pet,
group subsets of the multiple behavioral signs into one of multiple principal component behavioral groupings to generate principal component scores for each of the multiple principal component behavioral groupings, and
correlate the multiple principal component scores for each of the multiple principal component behavioral groupings to assign the individual pet to a population cluster associated with a type of separation-related problem, wherein the population cluster has a better fit for the separation-related problem than other population clusters associated with other types of separation-related problems.

16. The machine-learning system of claim 15, wherein the data store further includes instructions that, when executed, cause the system to report the population cluster, the separation-related problem, or both to the pet caregiver at the client device.

17. The machine-learning system of claim 15, wherein at least a portion of the instructions are generated using a training dataset, wherein the training dataset is derived from a population of pets of the same species as the individual pet.

18. The machine-learning system of claim 17, wherein the training dataset is used to find or optimize a machine-learning pipeline which returns accurate identification of the population cluster for the individual pet with at least 90% confidence.

19. The machine-learning system of claim 18, wherein the machine-learning pipeline includes a linearly fitted model utilizing tree-based pipeline optimization resulting in the use of a stochastic gradient decent algorithm.

20. The machine-learning system of claim 18, wherein the instructions are validated using a validation dataset derived from a second population of pets of the same species as the individual pet, wherein the validation dataset confirms the accuracy of the identification of the population cluster for the individual pet.

21. The machine-learning system of claim 20, wherein the instructions are validated using tree-based pipeline optimization and k-fold cross-validation.

22. The machine-learning system of claim 15, wherein the pet is a dog, and the principal component behavioral groupings include at least one of exit frustration (Ef), redirected frustration (Rf), social panic (Sp), elimination (E), reactive communication (Rc), immediate frustration (If), or noise sensitivity (Ns); and wherein the population clusters are each associated with a unique separation-related problem including at least one of exit frustration, redirected reactive, reactive inhibited, or boredom.

23. A non-transitory machine-readable storage medium having instructions embodied thereon, the instructions when executed by one or more processors, cause the one or more processors to perform to:
correlate the behavioral information with a plurality of binary sign scores related to the presence or absence of multiple behavioral signs related to the individual pet,
group subsets of the multiple behavioral signs into one of multiple principal component behavioral groupings to generate principal component scores for each of the multiple principal component behavioral groupings,
correlate the multiple principal component scores for each of the multiple principal component behavioral groupings to assign the individual pet to a population cluster associated with a type of separation-related problem, wherein the population cluster has a better fit for the separation-related problem than other population clusters associated with other types of separation-related problems, and
report the population cluster, the separation-related problem, or both to the pet caregiver at the client device.

24. The non-transitory machine-readable storage medium of claim 23, wherein at least a portion of the instructions are generated by machine-learning using a training dataset derived from a population of pets of the same species as the individual pet, and wherein the instructions are validated using a validation dataset derived from a second population of pets of the same species as the individual pet.

25. The non-transitory machine-readable storage medium of claim 23, wherein the pet is a dog, and wherein the principal component behavioral groupings include exit frustration (Ef), redirected frustration (Rf), social panic (Sp), elimination (E), reactive communication (Rc), immediate frustration (If), noise sensitivity (Ns), or a combination thereof, and wherein population clusters are each associated with a unique separation-related problem selected from exit frustration, redirected reactive, reactive inhibited, boredom, or a combination thereof.
